(19) United States
(12) Patent Application Publication
(10) Pub. No.: US 2006/0094790 A1
(43) Pub. Date: May 4, 2006

(54) Use of sphingosine kinase activator as skin disease treating agent and method for treating skin diseases using the same

(76) Inventors: Byeong-Deog Park, Cheongju (KR); Jong-Kyung Youm, Dajeon (KR); Hyung-Sub Gwak, Dajeon (KR); Mi-Jong Kwon, Dajeon (KR); Yong-Moon Lee, Dajeon (KR); Yoo-Hun Kim, Cheongju (KR); Hwan-Mook Kim, Dajeon (KR); Song-Kyu Park, Dajeon (KR); Ki-Ho Lee, Dajeon (KR); Chang-Woo Lee, Dajeon (KR); Myong-Lyoll Lee, Dajeon (KR)

Correspondence Address: Galgano & Burke, Suite 35, 300 Rabro Drive, Hauppauge, NY, 11788, US

(21) Appl. No.: 11/260,745

(22) Filed: Oct. 27, 2005

(30) Foreign Application Priority Data
Nov. 3, 2004 (KR) 10-2004-0088553

(51) Int. Cl. A61K 31/16

(52) U.S. Cl. 514/625

(57) ABSTRACT

Disclosed is a non-natural ceramide compound effective for a sphingosine kinase activator, and thus useful for a skin disease treating agent. The sphingosine kinase activator enhances production of sphingosine-1-phosphate to show various physiological activities provided by sphingosine-1-phosphate. The physiological activities include the effects of: controlling multiplication and differentiation of keratinocytes, multiplication of fibroblasts and collagen synthesis, resulting in treatment of wounds, recovery of damaged skin functions in atopic dermatitis and psoriasis; inhibiting wrinkles and skin irritation caused by ultraviolet rays, followed by improvement of wrinkles and inhibition of skin aging; and reducing skin atrophy, which is a typical side effect of local application steroids. Therefore, the sphingosine kinase activator is useful for a skin disease treating agent for treating skin wounds, wrinkles, atopic dermatitis, eczema, psoriasis, or skin atrophy caused by side effects of local application steroids.

### BACKGROUND OF THE INVENTION

1. Field of the Invention

The present invention relates to use of a sphingosine kinase activator as a skin disease-treating agent and a method for treating skin diseases using the same. More particularly, the present invention relates to use of a sphingosine kinase activator as a skin disease-treating agent, wherein the sphingosine kinase activator enhances biosynthesis of sphingosine-1-phosphate by the sphingosine kinase to show various physiological activities provided by sphingosine-1-phosphate, which includes: effects of inducing intracellular calcium movement and thus controlling multiplication and differentiation of keratinocytes in skin cells; multiplication of fibroblasts and collagen synthesis, resulting in treatment of wounds; recovery of damaged skin functions in atopic dermatitis and psoriasis; inhibition of wrinkles and skin irritation caused by ultraviolet rays, followed by improvement of wrinkles and inhibition of skin aging; and reduction of skin atrophy, which is a typical side effect of steroids. The present invention also relates to a method for activating the sphingosine kinase and a method for treating skin diseases using the above sphingosine kinase activator.

2. Description of the Prior Art

In general, sphingosine-1-phosphate (SIP) is known merely as one of the metabolic by-products of sphingolipids. However, according to a recent study, it is reported that the above compound has physiological activities to control various biological processes. More particularly, it is known that the above compound functions as a secondary signal transferring agent that controls multiplication and survival of cells, from the intracellular point of view, while functioning as a ligand for EDG (endothelial differentiation gene) receptors (EDG-1, 3, 5, 6, 8) that belong to G-protein coupled receptors, from the extracellular point of view (see Spiegel S. et al., Biochem. Biophys. Acta, 1484, 107-116, 2000).

Particularly, from the intracellular point of view, it is reported that sphingosine-1-phosphate causes calcium to move from internal depots into cytoplasm, independently from the calcium signal transfer system caused by 1,4,5-triphosphate, thereby forming various signal transfer paths resulting in multiplication of cells and inhibition of cell destruction. It is also reported that a competitive inhibitor against the sphingosine kinase prevents production of sphingosine-1-phosphate, inhibits calcium movement selectively, and affects multiplication, differentiation and survival of cells by various stimuli depending on the type of cell (see Spiegel S. et al., J. Leukoc. Biol., 65, 341-344, 1999).

Additionally, it is reported that sphingosine-1-phosphate, which is normally stored in platelets of the human body, is delivered to the site of a skin wound, so as to play an important role in treating wounds (see Lee et al., Am J Physiol Cell Physiol, 278, C612-C618, 2000). Further, it is reported that 1α,25-dihydroxyvitamin D₃, known as a sphingosine kinase activator, inhibits cell destruction of keratinocytes (see Manggau et al., J Invest Dermatol, 117, 1241-1249, 2001). In addition to the above, it is reported that sphingosine-1-phosphate plays a very important role in treating skin wounds, because the compound inhibits cell destruction for keratinocytes, enhances movement of cells, enhances multiplication of fibroblasts, and stimulates extracellular formation of matrix proteins (see Vogler et al., J Invest Dermatol, 120, 693-700, 2003).

Materials known to activate sphingosine kinase and to enhance biosynthesis of sphingosine-1-phosphate include 1α,25-dihydroxyvitamin D₃, PMS (phorbolmyristate acetate), N-formyl-methionyl-leucylphenylalanine, platelet-derived growth factors and nerve growth factors. However, only the 1α,25-dihydroxyvitamin D₃ is commercially available as a psoriasis treating agent, and the other materials have problems in that they are strongly toxic materials which cause cancer, or have difficulty in their synthesis. Although sphingosine 1-phosphate may be obtained by chemical synthesis, it is difficult to synthesize sphingosine-1-phosphate and such synthetic processes are not cost-efficient.

### SUMMARY OF THE INVENTION

Therefore, the present invention has been made in view of the above-mentioned problems. It is an object of the present invention to provide use of a sphingosine kinase activator, which enhances production of sphingosine-1-phosphate to provide physiological activities of sphingosine-1-phosphate efficiently, as a treating agent for treating skin diseases including skin wounds, wrinkles, atopic dermatitis, eczema, psoriasis and skin atrophy caused by side effects of local application steroids.

Another object of the present invention is to provide use of the sphingosine kinase activator in a composition for treating skin diseases, including skin wounds, wrinkles, atopic dermatitis, eczema, psoriasis and skin atrophy caused by side effects of local application steroids.

Still another object of the present invention is to provide a method for activating the sphingosine kinase.

Yet another object of the present invention is to provide a method for treating a patient suffering from skin diseases, including skin wounds, wrinkles, atopic dermatitis, eczema, psoriasis and skin atrophy caused by side effects of local application steroids, by using the above sphingosine kinase activator.

According to an aspect of the present invention, in order to accomplish the first object of the present invention, there is provided use of a sphingosine kinase activator, which is at least one selected from the group consisting of compounds represented by the following formulae 1 to 8, as a skin disease treating agent: 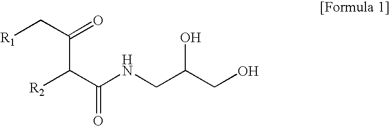
wherein each of R₁ and R₂ is a linear or branched C₄˜C₂₂ alkyl group. 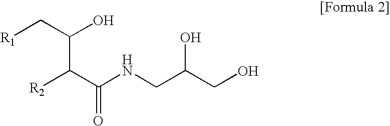
wherein each of R₁ and R₂ is a linear or branched C₄˜C₂₂ alkyl group. 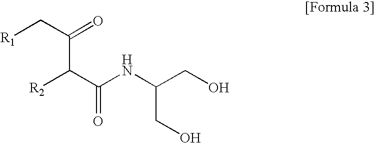
wherein each of R₁ and R₂ is a linear or branched C₄˜C₂₂ alkyl group. 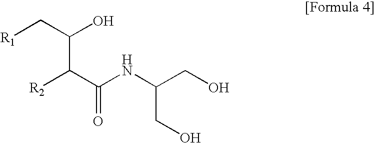
wherein each of R₁ and R₂ is a linear or branched C₄˜C₂₂ alkyl group. 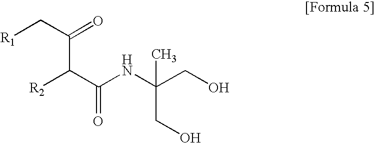
wherein each of R₁ and R₂ is a linear or branched C₄˜C₂₂ alkyl group. 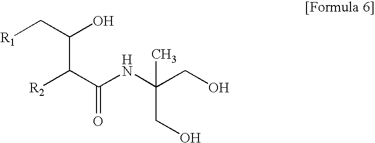
wherein each of R₁ and R₂ is a linear or branched C₄˜C₂₂ alkyl group. 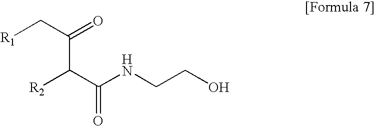
wherein each of R₁ and R₂ is a linear or branched C₄˜C₂₂ alkyl group. 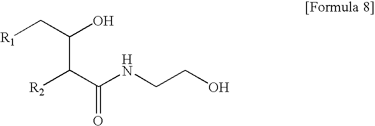
wherein each of R₁ and R₂ is a linear or branched C₄˜C₂₂ alkyl group.

According to another aspect of the present invention, in order to accomplish the second object of the present invention, there is provided use of at least one sphingosine kinase activator, selected from the group consisting of compounds represented by the above formulae 1 to 8, in a composition for treating skin diseases, including skin wounds, wrinkles, atopic dermatitis, eczema, psoriasis and skin atrophy caused by side effects of local application steroids, wherein the sphingosine kinase activator is used in an amount of 0.001 to 50.0 wt % based on the total weight of composition.

According to still another aspect of the present invention, in order to accomplish the third object of the present invention, there is provided a method for activating the sphingosine kinase, which comprises applying at least one sphingosine kinase activator selected from the group consisting of compounds represented by the above formulae 1 to 8 to the skin of a patient suffering from skin diseases, including skin wounds, wrinkles, atopic dermatitis, eczema, psoriasis and skin atrophy caused by side effects of local application steroids.

According to yet another aspect of the present invention, in order to accomplish the fourth object of the present invention, there is provided a method for treating skin diseases, which comprises applying an effective amount of at least one sphingosine kinase activator selected from the group consisting of compounds represented by the above formulae 1 to 8 to the skin of a patient suffering from skin diseases, including skin wounds, wrinkles, atopic dermatitis, eczema, psoriasis and skin atrophy caused by side effects of local application steroids.

According to the present invention, the sphingosine kinase activator and the skin disease treating agent comprising the same as active component are efficient for treating skin wounds, for alleviating, mitigating and treating atopic dermatitis, eczema and psoriasis conditions, for improving wrinkles, for preventing skin aging, and for inhibiting side effects caused by local application steroids.

The sphingosine kinase activator according to the present invention activates sphingosine kinase, so as to enhance biosynthesis of sphingosine-1-phosphate and to provide various physiological activities of sphingosine-1-phosphate.

The present inventors have found that the above sphingosine kinase activator inhibits multiplication of keratinocytes, enhances differentiation of keratinocytes, enhances multiplication of fibroblasts and stimulates collagen synthesis, and thus is highly efficient for treating wounds, inhibiting multiplication of keratinocytes and enhancing differentiation of keratinocytes in the real skin. Accordingly, we have demonstrated that the above sphingosine kinase activator provides the effects of recovering damaged skin functions in atopic dermatitis, eczema and psoriasis, inhibiting wrinkles and skin irritation caused by ultraviolet rays, so as to improve wrinkles and inhibiting skin aging, and inhibiting skin atrophy caused by local application steroids, so as to reduce side effects of steroids.

Although there is no particular limitation in amount of the sphingosine kinase activator in the skin disease treating agent according to the present invention, the sphingosine kinase activator is present preferably in an amount of 0.001 to 50 wt %, more preferably in an amount of 0.01 to 30 wt %, based on the total weight of the treating agent. When the sphingosine kinase activator is used in an amount beyond the above range, the treating agent cannot provide the desired effects to a sufficient degree or is not cost-efficient.

The treating agent comprising the sphingosine kinase activator as active component can be applied to any formulations for skins. More particularly, the treating agent may be formulated into the form of a toner, lotion, cream, essence, pack, powder, ointment, suspension, emulsion, spray, cosmetic solution, soap, shampoo, skin patch, gel, and so on. Additionally, the sphingosine kinase activator may be formulated in the form of a skin-contacting material such as a cosmetic product, detergent and fiber.

### BRIEF DESCRIPTION OF THE DRAWINGS

The above and other objects, features and advantages of the present invention will be more apparent from the following detailed description taken in conjunction with the accompanying drawings, in which:

FIGS. 1a to 1d are graphs each showing intracellular calcium movement induced by the sphingosine kinase activator according to the present invention, in a signal transfer system for providing physiological activities of the cells;

FIG. 2 is a photograph showing the results of polyacrylamide gel electrophoresis, which demonstrates the effects of the sphingosine kinase activator upon differentiation of keratinocytes;

FIGS. 3a to 3c are photographs each showing the effects of the sphingosine kinase activator upon calcium gradient in a skin horny layer, when evaluated in an acute disruption model using tape striping against the back of a hairless mouse;

FIGS. 4a and 4b are graphs showing the effect of the sphingosine kinase activator upon inhibition of wrinkles caused by ultraviolet rays, and a photograph of the real skin of a rat, respectively;

FIG. 5 is a photograph of mouse skin tissues, which shows the effect of the sphingosine kinase activator upon inhibition of skin atrophy caused by side effects of steroids; and

FIGS. 6a and 6b are a graph and photograph of a silicone replica, each showing the effect of the sphingosine kinase activator upon improvement of wrinkles around the eye in a clinical test to humans.

### DETAILED DESCRIPTION OF THE PREFERRED EMBODIMENTS

Reference will now be made in detail to the preferred embodiments of the present invention. It is to be understood that the following examples are illustrative only and the present invention is not limited thereto.

The sphingosine kinase activator used in the following examples are compounds represented by the above formulae 1 to 8, wherein R₁═R₂═C₆, which include N-(2,3-dihydroxypropyl)-2-hexyl-3-oxo-decanamide (referred to as ‘K6PC4’ hereinafter), N-(1,3-dihydroxyisopropyl)-2-hexyl-3-oxo-decanamide (referred to as ‘K6PC-5’ hereinafter), N-(2-methyl-1,3-dihydroxyisopropyl)-2-hexyl-3-oxo-decanamide (referred to as ‘K6PC-7’ hereinafter), and N-ethanol-2-hexyl-3-oxo-decanamide (referred to as ‘K6PC-9’ hereinafter).

First, in Examples 1 to 4, in vitro tests for K6PC4, K6PC-5, K6PC-7 and K6PC-9 are performed. In these examples, each of the above compounds is evaluated for intracellular calcium movement, sphingosine kinase activation capability, collagen synthesizing capability in fibroblasts and keratinocyte differentiation capability. After evaluation, it is determined that each compound has the effects of treating wounds, aiding recovery of skin barrier functions in treating atopic dermatitis, eczema and psoriasis, improving wrinkles, inhibiting skin aging, and treating skin atrophy caused by side effects of local application steroids.

### EXAMPLE 1

### Effect of Sphingosine Kinase Activator Upon Intracellular Calcium Movement

First, the following experiment for calcium movement was performed in order to determine whether the above sphingosine kinase activator compounds activate sphingosine-1-phosphate, so as to cause intracellular calcium movement, which is a typical activity specific to sphingosine-1-phosphate.

The sphingosine kinase activator compounds were determined for capability of inducing intracellular calcium movement by using a RBL-2H3 cell line, which is one of the typical cell lines showing intracellular calcium movement. The cells are cultured by using an RPMI 1640 culture medium and the cells were washed simultaneously with removal of the medium. Then, 10 μM of fura-2/Am and 250 μl M of sulfinpyrazone were added thereto, followed by incubation for 30 minutes. Cell pellets were obtained by centrifugal separation and the cell pellets were dispersed in Ca²⁺-free Locke's solution. The dispersion was divided into a unit of 1×10⁶ cells for use in treating samples. Next, the cells were introduced into a cuvet of a fluorescence microscope, each sphingosine kinase activator compound was added thereto, and intracellular calcium movement was observed by the fluorescence microscope (RF-5310PC, Spectrofluoro photometer, SHIMADZU). When intracellular calcium ions are detected, they cause fluorescence by bonding with fura-2. Therefore, a degree of calcium movement caused by a sample was evaluated by calculating a difference between the measurement of fura-2 (380 nm) bonded with calcium and that of non-bonded fura-2 (340 nm).

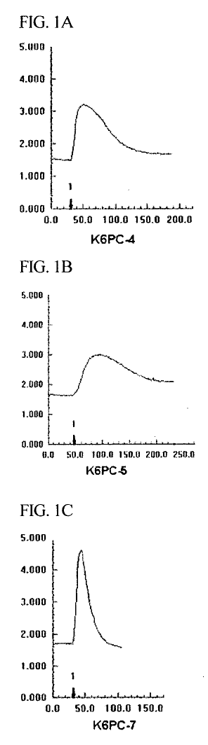

After the evaluation, as shown in FIGS. 1a to 1d, all of K6PC4, K6PC-5, K6PC-7 and K6PC-9 caused intracellular calcium movement. It can be seen that such calcium signal transfer is a mechanism of encouraging physiological activities of cells.

### EXAMPLE 2

### Effect for Activation of Sphingosine Kinase

The following test for activation of sphingosine kinase was performed to determine whether the effects of the above compounds upon intracellular calcium signal transfer as described in Example 1 were caused by activation of sphingosine kinase.

F9-12 cells were treated with 300 nM of PMA (phorbol microstate acetate) as positive control, and 50 μM of each of K6PC4 and K6PC-5 for 24 hours and collected. Then, activity of sphingosine kinase was measured as production of C₁₇-sphingosine-1-phosphate based on 50 μg of protein. Sphingosine-1-phospate was extracted from the collected cells by the steps of: (1) treating with trypsin-EDTA, (2) centrifugal separation at 1,500 rpm for 10 minutes, and (3) washing with PBS, followed by freeze-drying. Then, PBS was added to the freeze-dried product and the resultant product was treated with ultrasonic waves to destroy cells. Sphingosine-1-phosphate was determined by HPLC, and OPA (o-phthalaldehyde) reagent and boric acid buffer were added to the extracted sample and the mixture was reacted at room temperature for 20 minutes. For the HPLC quantitive analysis, fluorescence intensity was measured at each wavelength of 340 nm and 455 nm with a solution in 90% acetonitrile, and a ratio to the internal standard was calculated.

After the evaluation of sphingosine kinase activity, K6PC4 and K6PC-9 showed an increase in production of sphingosine-1-phosphate by about 30%, and K6PC-5 and K6PC-7 showed an increases in production of sphingosine-1-phosphate by about 46%, as shown in the following Table 1. Meanwhile, PMA used as positive control showed an increase of about 48%. Therefore, it can be seen that the above compounds according to the present invention serve as sphingosine kinase activators.

TABLE 1
Activation of Sphingosine Kinase

| | C17-SIP Production | |
|---|---|---|
| | (pmol/min/mg) | Standard Deviation |
| Control (non-treated) | 51.72 | 7.86 |
| PMA (positive control) | 76.76 | 6.15 |
| K6PC-4 | 67.69 | 5.51 |
| K6PC-5 | 75.59 | 2.06 |
| K6PC-7 | 75.51 | 10.0 |
| K6PC-9 | 66.72 | 5.4 |

### EXAMPLE 3

### Effect for Collagen Synthesis in Fibroblasts

The following experiment was performed by using fibroblasts in order to evaluate the effect of sphingosine kinase activator upon collagen synthesis.

Enhancement of collagen synthesis upon application to the human body contributes to treatment of wounds, treats wrinkles caused by skin aging and inhibits skin atrophy occurring as a atypical side effect of steroids.

Each of K6PC4, K6PC-5, K6PC-7 and K6PC-9 was dissolved in DMSO at a concentration of 0.3 and 1.0 μg/ml. The solutions were used as samples and analyzed for collagen synthesis after incubation for 72 hours. 72 hours after the treatment of the sample, culture solution was discarded, cells were washed with serum-free DMEM three times, and cells were cultured again by using flesh serum-free DMEM. After incubation, supernatant in each well was combined and analyzed for the amount of PICP (procollagen type I C-peptide) by using a collagen measuring kit. The standard solution contained in the collagen measuring kit was diluted with a sample and absorption at 450 nm was measured to construct a standard concentration curve (see the following Table 2).

TABLE 2
Dilution Ration of Standard Solution with Sample Dilution for Construction of Standard Concentration Curve

| Final Concentration (ng/ml) | 0 | 10 | 20 | 40 | 80 | 160 | 320 | 640 |
|---|---|---|---|---|---|---|---|---|
| Sample Dilution | 400 μl | 393.75 μl | 387.5 μl | 375 μl | 350 μl | 300 μl | 200 μl | — |
| Standard Solution | — | 6.25 μl | 12.5 μl | 25 μl | 50 μl | 100 μl | 200 μl | 400 μl |
| (640 ng/ml) | | | | | | | | |

To an antibody-coated microtiter plate comprising a primary collagen antibody applied unifomrily thereto, 100 μl of an antibody-POD conjugate solution and the cell supernatant collected as described above were added, followed by incubation at 37° C. for 3 hours, to induce an antigen-antibody reaction. Then, the reaction mixture was subjected to washing and color developing. After the reaction, the reaction mixture developed a yellow color, wherein the yellowness depended on reaction degrees. Also, 96 well plates developing a yellow color were determined at 450 nm by using a microtiter plate reader.

After the evaluation for collagen synthesis in fibroblasts, collagen synthesis was increased by about 1.7 times in the case of K6PC-4, about 2.4 times in the case of K6PC-5, about 1.9 times in the case of K6PC-7, and about 2.0 times in the case of K6PC-9, as compared to the non-treated control. The results are shown in the following Table 3.

Therefore, it can be seen that when the above compounds are applied to the human body, it is possible to increase collagen synthesis, and thus to treat wounds, improve wrinkles and to reduce side effects caused by steroids.

TABLE 3
Amount of PICP produced in fibroblasts (average ± SEM)

| Concentration | Control | Samples | | | |
|---|---|---|---|---|---|
| (μg/ml) | (non-treated) | K6PC-4 | K6PC-5 | K5PC-7 | K6PC-9 |
| 0.0 | 47.1 ± 9.7 | | | | |
| 0.3 | | 62.01 ± 2.5 | 111.2 ± 3.9 | 90.3 ± 12.5 | 95.0 ± 5.2 |
| 1.0 | | 80.51 ± 4.4 | 96.0 ± 5.6 | 87.3 ± 14.9 | 86.7 ± 8.2 |

### EXAMPLE 4

### Effect for Inducing Differentiation of Keratinocytes

Effects of the sphingosine kinase activator according to the present invention upon inhibition of cell multiplication and differentiation were evaluated by using keratinocytes.

Keratinocytes form the outermost layer of the skin and play a very important role in skin moisturizing and protecting functions. It is preferable to inhibit excessive growth of keratinocytes and cell destruction and to enhance differentiation of keratinocytes. Excessive multiplication of keratinocytes results in abnormal extension of the stratum corneum, followed by roughening and thickening of the skin. Additionally, abnormal differentiation of keratinocytes inhibits normal skin barrier functions, and thus may cause various troubles, including skin dryness, atopic dermatitis and psoriasis.

In order to evaluate the effect of the above compound according to the present invention upon differentiation of keratinocytes, each of K6PC4, K6PC-5, K6PC-7 and K6PC-9 was dissolved in DMSO at a concentration of 10 μM and used as sample. Evaluation was performed by using the western blotting method. As differentiation markers, involucrin and keratine-1, which were differentiation markers of keratinocytes, were measured. Next, 48 hours after the treatment of samples, the culture solution was discarded and the cells were washed with PBS and collected by filtering. The collected cells were washed again and subjected to centrifugal separation to remove supernatant. The cells were dissolved in a solvent and subjected to centrifugal separation at 12,000 rpm for 10 minutes, thereby removing cell membranes, etc. Protein concentration was determined by the Bradford method. Proteins were separated by mini gel type SDS-PAGE (polyacrylamide gel electrohoresis) and transferred to a PVDF (polyvinylidene fluoride) membrane at 100V for 1 hour, so that gel-like proteins were subjected to blotting with a transfer membrane. Then, the membrane was colored with Ponceau S solution to determine whether transfer was accomplished or not. The membrane was blocked by using TTBS (TBS+0.1% Tween 20) solution containing 5% non-fat dried milk. In order to check the amount of involucrin as differentiation marker of keratinocytes, primary antibody involucrin (Neomarkers Co.) was diluted at a ratio of about 1/200 to 1.400, and keratin-1 (Covance Co.) was diluted at a ratio of about 1/1000. Reactions were performed overnight at 4° C. As secondary antibody, anti-mouse IgG and anti-rabbit IgG combined with horseradish peroxidase (HRP) was diluted at a ratio of 1:2000. Then, the secondary antibody was bonded to the primary antibody by making them react at room temperature for 1 hour. The membrane was washed with TTBS three times, reacted with an ECL substrate (Amersham Co.) for 1-3 minutes, and exposed to X-ray films.

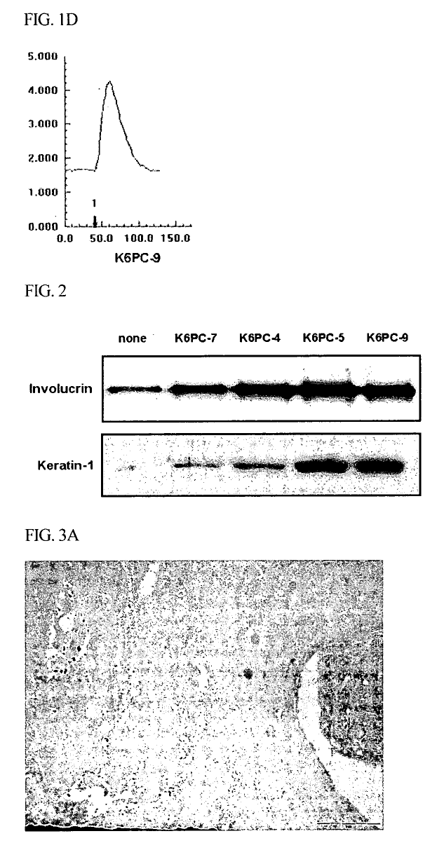

After the evaluation of effect for differentiation of keratinocytes, all of K6PC-4, K6PC-5, K6PC-7 and K6PC-9 expressed the differentiation markers, as shown in FIG. 2. Particularly, better results were obtained in the case of involucrine. Therefore, it can be seen that the above compounds enhance differentiation of keratinocytes. As demonstrated by such increased expression of differentiation markers compared to the control, the compounds according to the present invention can enhance differentiation of keratinocytes, resulting in rapid recovery of the skin barrier functions.

Hereinafter, an in vivo test for K6PC-5, which represents for the compounds according to the present invention (i.e. K6PC4, K6PC-5, K6PC-7 and K6PC-9), was performed through the following Examples 5 to 7. After the in vivo test, K6PC-5 showed excellent effects of recovering a calcium gradient in the epidermis, differentiating keratinocytes on the epidermis, inhibiting wrinkles cause by ultraviolet rays and reducing side effects of steroids.

### EXAMPLE 5

### Effect for Recovery of Calcium Gradient on Epidermis

To determine the effect of recovering skin barrier effects obtained by the compound according to the present invention, the compound was evaluated for the effect of recovering a calcium ion gradient in the epidermis.

The calcium gradient in the epidermis plays very important role in maintaining homoeostasis of the skin barrier function. For example, when a hairless mouse is subjected to acute disruption on its back by tape stripping, the calcium ion gradient in the epidermis is lost. Therefore, it is possible to evaluate the effect of a test sample upon recovery of a damaged skin barrier by observing recovery of the calcium gradient loss in an acute disruption model.

In the following test, K6PC-5 according to the present invention was evaluated for the effect upon variations in calcium gradient in an acute disruption model.

Tissues non-treated with the sample were provided as control, and tissues were collected right after the tape striping, and 3, 6 and 24 hours after the tape stripping. Then, the tissues treated with K6PC-5 (1.0% in PEG:EtOH=7:3) were compared with controls, which are treated by calcium ion capture cell chemical dyeing. More particularly, each freshly collected tissue sample was fixed with a fixing solution comprising 2% glutaraldehyde, 2% formaldehyde, 90 mM potassium oxalate and 1.4% sucrose and refrigerated at 4° C. Then, each sample was subjected to calcium ion capture cell chemical dyeing in order to observe calcium ions. One drop of the refrigerated fixing solution was applied to a three dimensional microscope, cut finely into a size of 0.5 mm×3, and then fixed on crashed ice overnight. The fixing solution was discarded, and the sample was mixed with 1 ml of 4% OSO₄ and 3 ml of 2% potassium pyroantimonate stock solution, and further fixed with the fixing solution by placing it on ice for 2 hours. Then, all of the fixed tissues were washed with cold distilled water (pH 10) for 10 minutes, and dewatered, formatted and dyed in a conventional manner. The sample provided as described above was observed for all layers of the epidermis under a transmission electron microscope.

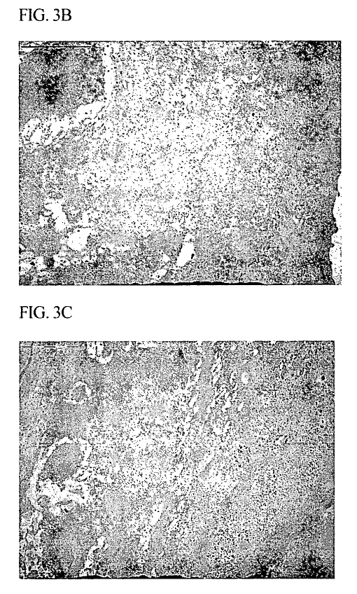

After performing the calcium ion capture cell chemical dyeing in the acute disruption model, calcium loss right after the tape stripping of the sample treated with K6PC-5 began to be recovered, from 3 hours after the treatment, to form the normal calcium gradient rapidly, as compared to the control. FIG. 3a is a photograph illustrating the calcium loss in the epidermis right after the tape stripping. FIG. 3b is a photograph taken after the lapse of 6 hours as control. FIG. 3c is a photograph showing the result obtained 6 hours after the treatment with K6PC-5.

The above results indicate that the sphingosine kinase activator according to the present invention enhances rapid recovery of the skin barrier functions, because calcium functions as important signal transfer material in a damaged skin barrier. Therefore, it can be seen from the above in vivo test that the sphingosine kinase activator according to the present invention has the effects of treating wounds, treating atopic dermatitis, eczema and psoriasis, and preventing skin from aging.

### EXAMPLE 6

### Evaluation for Effects of Inhibiting Wrinkles and Skin Aging

To evaluate the effects of the sphingosine kinase activator according to the present invention upon inhibition of wrinkles and aging, a rat model, in which wrinkles are induced by ultraviolet rays, is used to determine the effects of inhibiting wrinkles and preventing side effects caused by ultraviolet rays. Generally, continuous exposure to UV causes wrinkles and side effects such as sun burn and skin irritation, resulting in stimulation of skin aging.

To perform the evaluation, an SD rat with three wrinkles is irradiated with UVB under an intensity of 130 mJ/cm² at its rear leg three ties for 6 weeks. Then, 10 μl (1% in 80% EtOH) of K6PC-5 was applied to the skin of rear leg right after each time of UV irradiation, UV irradiation being performed 5 times per week during 6 weeks from the start day of the UV irradiation. Nine weeks after the treatment, the rat was anesthetized with albutin and the wrinkles were photographed. Additionally, wrinkle-forming portions were replicated by using an exafine hydrophilic vinyl polysiloxane impression material. The replicated images were analyzed quantitively for shadow images by using an image analyzer.

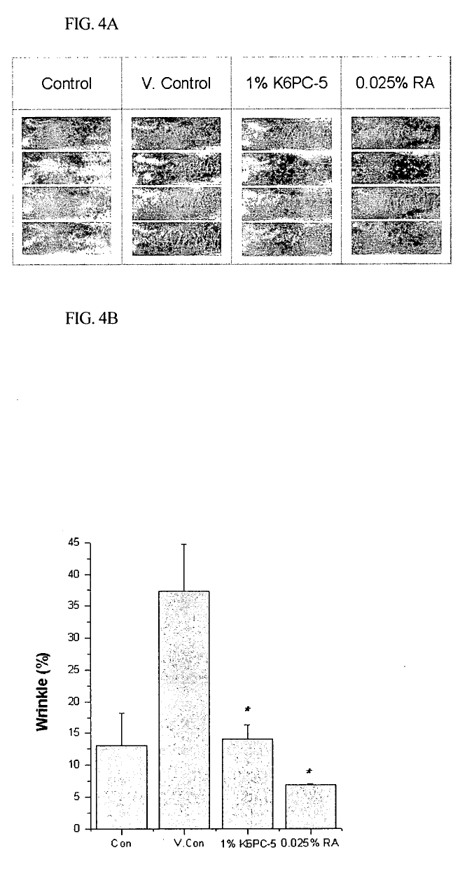

After the evaluation for the effects of inhibiting wrinkles and skin aging, the vehicle control (VC) irradiated with UV caused a significant amount of wrinkles, compared to the control non-irradiated with UV, as shown in FIG. 4a illustrating the results of the evaluation for inhibition of wrinkles. When compared to the VC, K6PC-5 inhibited wrinkles by about 63%. Additionally, as shown in the skin photograph of FIG. 4b, the sample treated with K6PC-5 showed a decrease in erythema (a typical side effect caused by UV) compared to the VC. Therefore, it can be seen that the sphingosine kinase activator according to the present invention is effective for improving wrinkles and preventing skin aging, as demonstrated the above results indicating inhibition of wrinkles and erythema that are typical side effects caused by UV.

### EXAMPLE 7

### Effect for Inhibiting Side Effects Caused by Steroids

To evaluate effect of the sphingosine kinase activator upon inhibition of steroid side effects, a steroid was applied to a hairless mouse. Typically, side effects caused by long-term or excessive dose of steroids include skin atrophy expressed by thinning of skin and weakening of skin functions, and a rebound phenomenon including reoccurrence of conditions caused by stopping use of steroids. It is reported that the main cause for such side effects is inhibition of fibroblast activity and a decrease in collagen production (S. Hammer et al., J. Cell. Biochem, 91, 840-851, 2004), Therefore, it is expected that the compound according to the present invention enhances collagen synthesis and differentiation of keratinocytes, and thus inhibits such side effects caused by steroids.

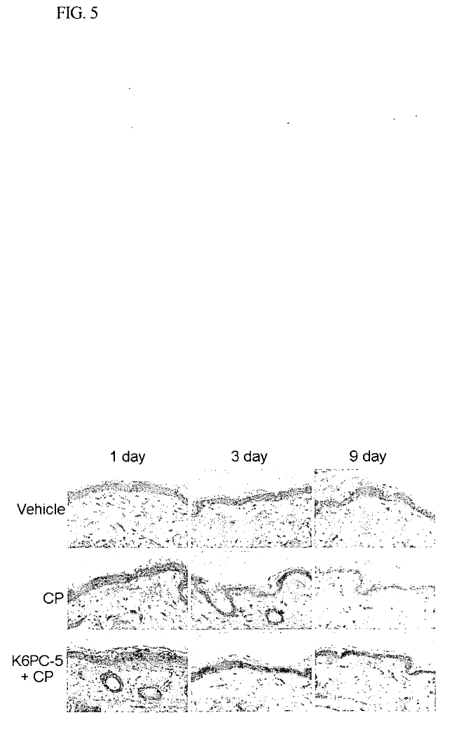

To perform a test, a steroid, i.e. 0.05% chlobetason-17-propionate, and K6PC-5 according to the present invention (1.0% in PEG:EtOH=7:3) were applied to a hairless mouse and changes in the skin were observed. The treating agents were applied to the back of a hairless mouse 9 times per day and the tissue was collected. Then, the epidermis and dermis were observed by carrying out the H & E staining method (hematoxylin and eosin staining) known to one skilled in the art. After the test, as shown in FIG. 5, the control free from steroids showed little change in the epidermis and dermis. The group, to which chlobetason-17-propionate was applied alone, showed significant thinning of the epidermis and abnormal changes in the dermis. However, the group treated with chlorbetason-17-propionate combined with K6PC-5 showed significant inhibition of side effects caused by steroids in a similar manner to the control. Therefore, it can be seen that the compound according to the present invention inhibits skin atrophy, which is a typical side effect caused by steroids.

### EXAMPLE 8

### Evaluation for Safety against Skin Irritation

To determine the safety of the sphingosine kinase activator according to the present invention when applying it to the human body as skin treating agent, both a toxicity test in animals and an application test to the human body were performed.

To perform this, a single dose oral toxicity test using rats, skin irritation test using rabbits, skin sensitization test using guinea pigs and an ophthalmic mucous membrane irritation test using rabbits were performed as toxicity tests for K6PC-5 in animals. Those tests were performed based on “Toxicity Test Standards for Pharmaceutical Products” disclosed by the Korea Food & Drug Administration. Additionally, an application test for K6PC-5 to the human body was carried out by using 30 subjects (average age: 25.8). After the tests, as shown in the following Table 4, only a slight skin irritation was observed in the skin irritation test using rabbits. However, when evaluating the overall results obtained from the other toxicity tests and human body application test, there is no problem in safety of the compound according to the present invention.

TABLE 4
Evaluation for Safety to Skin Irritation

| Test Item | Results |
|---|---|
| Single dose oral toxicity test (toxicity test) | No irritation |
| Skin irritation test using rabbits | Slight irritation |
| Skin sensitization test using guinea pigs | No irritation |
| Ophthalmic mucous membrane irritation test using | No irritation |
| rabbits | |
| Human body application test (1% K6PC-5) | No irritation |
| Human body application test (10% K6PC-5) | No irritation |

In the following Example 9, K6PC-5, which represents for K6PC4, K6PC-5, K6PC-7 and K6PC-9, was evaluated for the effect of inhibiting wrinkles through a clinical test. Additionally, the test in Example 9 aims to demonstrate clinical availability of the results obtained from the above in vivo and in vitro tests in human subjects.

### EXAMPLE 9

### Evaluation for Effect of Improving Eye Wrinkles in Human Subjects

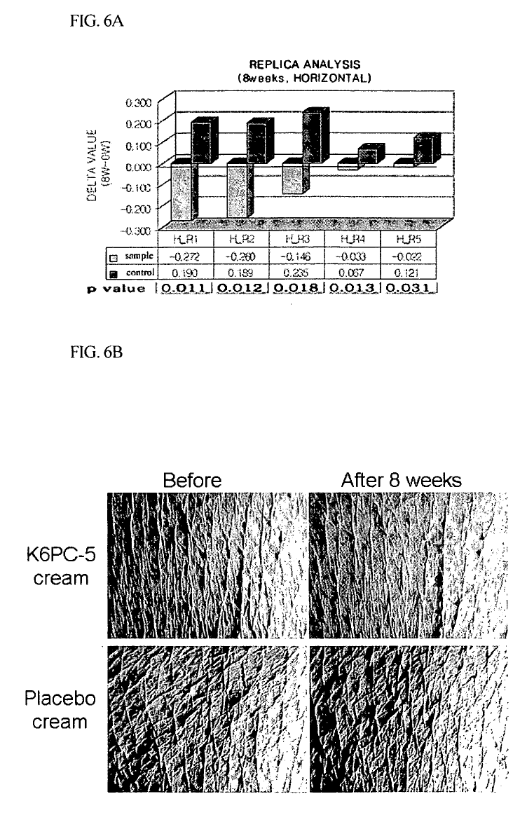

In this example, effects of improving wrinkles were evaluated in 32 subjects (average age: 46.7). During the total period of 8 weeks, a cream containing 1% of K6PC-5 and cream containing no K6PC-5 (control) were applied around both eye rims and then instrumental evaluation was performed. A silicone replica for the eye tail part of a subject was formed, and the replica was irradiated with light at an angle. Then, a shading degree formed by wrinkles in the replica was photographed by a CCD camera, and the image was determined for a wrinkling degree by using a computer image analysis system. More particularly, a program of Skin Visiometer SV 600 available from C+K Co. (Germany) was used for determination, wherein wrinkle parameters are expressed as R1 through R5 values. R1, R2 and R3 represent deep wrinkles and R4 and R5 represent shallow wrinkles. The results are shown in FIGS. 6a and 6b. FIG. 6a shows the effect of K6PC-5 upon a statistically significant improvement in the condition of wrinkles, as demonstrated by comparing the results obtained after the treatment with K6PC-5-containing cream for 8 weeks to the control (P<0.05, t-test). FIG. 6b is a photograph showing a real silicone replica, wherein improvement in the condition of wrinkles can be observed by the naked eyes. Additionally, according to catechetical and ocular inspection of dermatologists after the treatment with K6PC-5-containing cream for 4 weeks and 8 weeks, there is no skin irritation or hypersensitive reaction.

Therefore, the sphingosine kinase activator according to the present invention shows consistent results in the above in vitro test, in vivo test and clinical test.

Formulation 1: Emollient Cream

A moisturizing agent was added to purified water and heated to 70° C. K6PC-5 and oil phase components were dissolved by heating, and an emulsifier, preservative, or the like were added thereto, followed by heating to 70° C. The oil phase was added to the above aqueous phase. Then, emulsified particles were homogenized by using a homomixer, followed by deaerating, filtering and cooling.

TABLE 6

| Function | Ingredients | Amount (%) |
|---|---|---|
| Main component | K6PC-5 | 1.0 |
| Oil phase | Cetostearyl alcohol | 6.0 |
| components | Stearic acid | 2.0 |
| | Lanolin | 4.0 |
| | Squalane | 9.0 |
| | Octyldodecanol | 10.0 |
| Moisturizing agent | 1,3-butylene glycol | 3.0 |
| | Glycerin | 2.0 |
| Emulsifier | POE(25) cetyl alcohol ether | 3.0 |
| | Glycerin monostearate | 2.0 |
| Preservative | Propyl paraben | q.s. |
| | Methyl paraben | q.s. |
| | Purified Water | balance |

Formulation 2: Ointment for External Use

K6PC-5 and oil phase components were dissolved by heating, and an emulsifier, preservative, or the like was added thereto, followed by adjustment of the temperature to 70° C. The resultant mixture was mixed homogeneously by using a homomixer, followed by deaerating, filtering and cooling.

TABLE 7

| Function | Ingredients | Amount (%) |
|---|---|---|
| Main component | K6PC-5 | 1.0 |
| Oil phase | Petrolatum | balance |
| components | Cetostearyl alcohol | 2.0 |
| | Lanolin | 3.0 |
| | Squalane | 3.0 |
| Emulsifier | Ceteareth-20 | 3.0 |
| Preservative | Propyl paraben | q.s. |
| | Methyl paraben | q.s. |

Formulation 3: Moisturizing Lotion

A moisturizing agent was added to purified water and heated to 70° C. K6PC-5 and oil phase components were dissolved by heating, and an emulsifier, preservative, or the like were added thereto, followed by heating to 70° C. The oil phase was added to the above aqueous phase, and the resultant mixture was mixed homogeneously by using a homomixer, followed by deaerating, filtering and cooling.

TABLE 8

| Function | Ingredients | Amount (%) |
|---|---|---|
| Main component | K6PC-5 | 1.0 |
| Oil phase | Cetostearyl alcohol | 1.0 |
| components | Bees wax | 0.5 |
| | Vaselin | 2.0 |
| | Squalane | 6.0 |
| | Dimethylpolysiloxane | 2.0 |
| Emulsifier | POE(10) monooleate | 1.0 |
| | Glycerol monostearate | 1.0 |
| Moisturizing agent | Glycerin | 4.0 |
| | 1,3-butylene glycol | 4.0 |
| Preservative | Propyl paraben | q.s. |
| | Methyl paraben | q.s. |
| Others | 1% Aqueous Hyaluronic | balance |
| | acid solution | |
| | Purified Water | |

As can be seen from the foregoing, the sphingosine kinase activator according to the present invention enhances production of sphingosine-1-phosphate, and thus permits various physiological effects of sphingosine-1-phosphate to be utilized efficiently. More particularly, the skin disease treating agent comprising the sphingosine kinase activator according to the present invention as active component enhances collagen synthesis in fibroblasts, enhances differentiation of keratinocytes, and allows an abnormal calcium gradient in the epidermis to be recovered into a normal calcium gradient promptly, resulting in recovery of the skin barrier functions. Therefore, the skin treating agent provides the effects of treating wounds, recovering damaged skin functions in atopic dermatitis, eczema and psoriasis, inhibiting wrinkle formation caused by ultraviolet rays, improving the condition of wrinkles in the eye rims, and preventing skin aging. Further, the skin-treating agent inhibits skin atrophy caused by side effects of steroids, and thus is useful for an agent for alleviating side effects caused by steroids.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. Use of a sphingosine kinase activator, which is at least one selected from the group consisting of compounds represented by the following formulae 1 to 8, as a skin disease treating agent for treating skin diseases, including skin wounds, wrinkles, atopic dermatitis, eczema, psoriasis and skin atrophy caused by side effects of local application steroids: 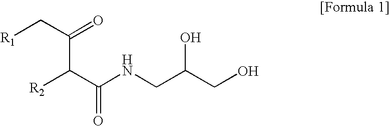
wherein each of R₁ and R₂ is a linear or branched C₄˜C₂₂ alkyl group. 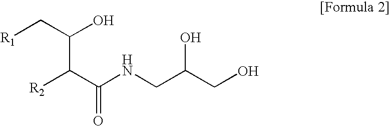
wherein each of R₁ and R₂ is a linear or branched C₄-C₂₂ alkyl group. 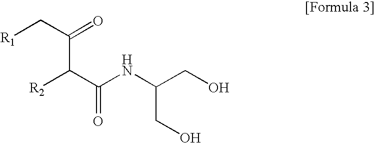
wherein each of R_{1 and R}₂ is a linear or branched C₄˜C₂₂ alkyl group. 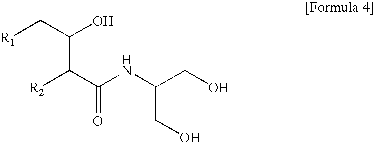
wherein each of R₁ and R₂ is a linear or branched C₄-C₂₂ alkyl group. 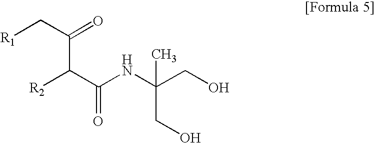
wherein each of R₁ and R₂ is a linear or branched C₄˜C₂₂ alkyl group. 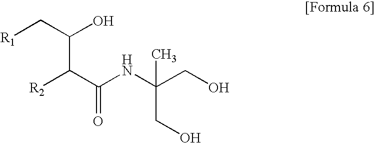
wherein each of R₁ and R₂ is a linear or branched C₄˜C₂₂ alkyl group. 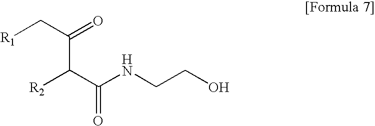
wherein each of R₁ and R₂ is a linear or branched C₄˜C₂₂ alkyl group. 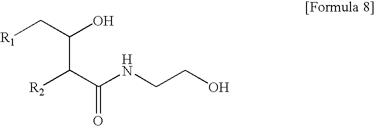
wherein each of R₁ and R₂ is a linear or branched C₄˜C₂₂ alkyl group.

2. The use according to claim 1, wherein each of R₁ and R₂ in the above formulae 1 through 8 is C₆.

3. The use according to claim 1, wherein the compound is at least one selected from the group consisting of: N-(2,3-dihydroxypropyl)-2-hexyl-3-oxo-decanamide), N-(1,3-dihydroxyisopropyl)-2-hexyl-3-oxo-decanamide, N-(2-methyl-1,3-dihydroxyisopropyl)-2-hexyl-3-oxo-decanamide, and N-ethanol-2-hexyl-3-oxo-decanamide.

4. Use of the sphingosine kinase activator as defined in claim 1 in a composition for treating skin diseases, including skin wounds, wrinkles, atopic dermatitis, eczema, psoriasis and skin atrophy caused by side effects of local application steroids, wherein the sphingosine kinase activator is used in an amount of 0.001 to 50.0 wt % based on the total weight of composition.

5. Use of the sphingosine kinase activator as defined in claim 2 in a composition for treating skin diseases, including skin wounds, wrinkles, atopic dermatitis, eczema, psoriasis and skin atrophy caused by side effects of local application steroids, wherein the sphingosine kinase activator is used in an amount of 0.001 to 50.0 wt % based on the total weight of composition.

6. Use of the sphingosine kinase activator as defined in claim 3 in a composition for treating skin diseases, including skin wounds, wrinkles, atopic dermatitis, eczema, psoriasis and skin atrophy caused by side effects of local application steroids, wherein the sphingosine kinase activator is used in an amount of 0.001 to 50.0 wt % based on the total weight of composition.

7. A method for activating sphingosine kinase, which comprises applying the sphingosine kinase activator defined in claim 1 to the skin of a patient suffering from skin diseases, including skin wounds, wrinkles, atopic dermatitis, eczema, psoriasis and skin atrophy caused by side effects of local application steroids.

8. A method for activating sphingosine kinase, which comprises applying the sphingosine kinase activator defined in claim 2 to the skin of a patient suffering from skin diseases, including skin wounds, wrinkles, atopic dermatitis, eczema, psoriasis and skin atrophy caused by side effects of local application steroids.

9. A method for activating sphingosine kinase, which comprises applying the sphingosine kinase activator defined in claim 3 to the skin of a patient suffering from skin diseases, including skin wounds, wrinkles, atopic dermatitis, eczema, psoriasis and skin atrophy caused by side effects of local application steroids.

10. A method for treating skin diseases, which comprises an effective amount of the sphingosine kinase activator as defined in claim 1, to the skin of a patient suffering from skin diseases, including skin wounds, wrinkles, atopic dermatitis, eczema, psoriasis and skin atrophy caused by side effects of local application steroids.

11. A method for treating skin diseases, which comprises an effective amount of the sphingosine kinase activator as defined in claim 2, to the skin of a patient suffering from skin diseases, including skin wounds, wrinkles, atopic dermatitis, eczema, psoriasis and skin atrophy caused by side effects of local application steroids.

12. A method for treating skin diseases, which comprises an effective amount of the sphingosine kinase activator as defined in claim 3, to the skin of a patient suffering from skin diseases, including skin wounds, wrinkles, atopic dermatitis, eczema, psoriasis and skin atrophy caused by side effects of local application steroids.
